(19) United States
(12) Patent Application Publication
(10) Pub. No.: US 2023/0044000 A1
(43) Pub. Date: Feb. 9, 2023

(54) SYSTEM AND METHOD USING AI MEDICATION ASSISTANT AND REMOTE PATIENT MONITORING (RPM) DEVICES

(71) Applicant: RxCap, Inc., Lynnfield, MA (US)

(72) Inventors: Sreeram K. Ramakrishnan, Lynnfield, MA (US); David MacVittie, Washington, DC (US); Michael Furlotti, Ross, CA (US); Ram Subramanian, Fremont, CA (US)

(21) Appl. No.: 17/884,396

(22) Filed: Aug. 9, 2022

Related U.S. Application Data
(60) Provisional application No. 63/230,963, filed on Aug. 9, 2021.

(51) Int. Cl. G16H 20/13; G16H 40/67; G16H 80/00

(52) U.S. Cl. CPC G16H 20/13; G16H 40/67; G16H 80/00

(57) ABSTRACT

A system for remotely managing a medication regimen includes a communication device usable by a target individual, a peripheral device that obtains biometric information of the target individual, an interactive medication dispenser, and a processor. The processor causes the communication device to enable a conversation interface associated with a virtual assistant that provides information, queries, and directions to a target individual, receives input from the target individual, information from the medication dispenser, and biometric information from the peripheral device, determines a status of management of the medication regimen based on the received data, determines a response based on the determined status, and transmits the response.

### BACKGROUND OF THE INVENTION

This invention relates to an automated system, bot, and method to promote medication adherence and perform remote patient monitoring (RPM).

With advancing technology and increasing use of that technology, such as smart phones, an increasing number of users now access information and services via websites or downloaded client applications provided by respective service providers. Such remote communication provides numerous benefits to both the service providers and the end users as compared to in person or over the phone communications, including the ability to offer information and services to end users at any time of day and without the cost associated with providing a human representative. Increasingly, service providers employ a “virtual assistant” to act as an interface between end users and the information on the service provider site. The virtual assistant can be in the form of a “bot,” a software application that is programmed to do certain tasks without specific instructions from humans. Some virtual assistants or bots can embody a human representative of the service provider displayed on a website, client application, or the like, and can also include an interface (e.g., a text box) that allows users to input queries, and the service provider or a third party can identify the contents of the user's query and provide a response. These virtual assistants or bots act as an effective interface that allows users to seek information and services of interest while still allowing service providers to realize cost savings associated with providing information online rather than via a human representative.

Now, consider the healthcare industry. Patients afflicted with acute or chronic illnesses may not find a cure for the condition despite ongoing treatment. Some patients may receive medical treatment including medication prescribed by a physician to relieve symptoms or prevent the illness from worsening. Some patients may need acute or limited time treatment care after release from hospital inpatient treatment, while the patient is still recovering. Treatment of some illnesses may require medications that require critical adherence to time of intake because of a narrow therapeutic window to improve efficacy or avoid toxicity. Some patients may experience loss of short term memory (for example, forgetfulness in early dementia, in an elderly or Alzheimer patient), or physically debilitating conditions, and hence, may be dependent on homecare without the benefit of expensive daily licensed nursing services.

Some medications used to treat chronic or acute illness may be expensive. The therapeutic effect of some medicine prescribed to treat a patient's chronic or acute illness may be limited by the patient's adherence to the dosing protocol prescribed by the patient's doctor. Elderly patients simply taking multiple drugs or patients with debilitating mental or physical illness (for example, diseases like Alzheimer's, Parkinson's, dementia, or multiple sclerosis) may not remember or be physically able to take drugs on time from multiple bottles traditionally dispensed by pharmacies.

In various examples, a patient's adherence to the dosing protocol prescribed by the patient's doctor may be a crucial component of caring for the patient's illness. Lack of adequate medication adherence may result in preventable disease progression and unnecessary expense.

Furthermore, during a dosing protocol, certain side effects may arise they may be harmful to the patient or adversely affect the treatment. Also, the dosing protocol may not be effective and may need to be adjusted or titrated or ceased altogether. This may entail switching medications or other therapy, but may be difficult to analyze if the patient is remote.

### SUMMARY OF THE INVENTION

In order to support targeted individuals on their healthcare journeys, focusing first on medication adherence and remote patient monitoring, an adherence bot is provided for use as a system with external devices, such as a smart cap, a hub, a blood pressure reader, or a scale, and can use Internet of Things (IoT) connectivity. A bot is a software application that is programmed to do certain tasks without specific instructions from humans. The adherence bot of the present invention is preferably automated and runs according to programmed instructions without a human user. The adherence bot will identify through analysis of aggregated data (from general population data and a targeted individual's own remote patient monitoring (RPM) devices and individual interactions) to assist the individual in maximizing his or her medication adherence. The bot will aid in identifying high risk individuals who would most likely need more coaching/assistance/interactions/touch points and tailor the interactions with the individual to help in improving and maintaining high levels of adherence/compliance. Additionally, the system can direct or focus caregivers (physicians, prescribers, pharmacists, healthcare entities) to provide more impactful attention to those individuals that are at risk of low adherence/compliance. Furthermore, the bot can determine whether the dosing protocol is effective and whether it may need to be adjusted or ceased altogether. This may entail titrating the medication, switching medications, or suggesting other therapy. After such adjustment, the adherence bot can continue performing monitoring and provide further recommendations for adjustment as necessary.

In one implementation, the adherence bot employs a conversation user interface to convey a representation of a conversation between the virtual healthcare assistant and the target individual (patient, user). The conversation UI presents a series of dialog representations, such as dialog bubbles, which include user-originated dialog representations associated with input from a user (verbal, textual, or otherwise) and device-originated dialog representations associated with response from the bot.

According to one aspect of the present invention, a system for remotely managing a medication regimen, includes a communication device usable by a target individual, the communication device communicating over a network; at least one peripheral device that obtains biometric information regarding the target individual, the at least one peripheral device communicating over the network to transmit the biometric information; a medication dispenser usable by the target individual, the medication dispenser communicating over the network to transmit information regarding the medication in the medication dispenser; and at least one processor communicating with the communication device, the at least one peripheral device, and the medication dispenser over the network. The at least one processor can execute computer-executable instructions to cause the communication device to enable a conversation interface associated with a virtual assistant, the virtual assistant providing at least one of information, queries, and directions to the target individual through the conversation interface; receive input from the target individual through the conversation interface; receive the information regarding the medication from the medication dispenser; receive the biometric information regarding the target individual from the at least one peripheral device; determine a status of management of the medication regimen based on at least one of the input from the target individual, the information regarding the medication from the medication dispenser, and the biometric information regarding the target individual from the at least one peripheral device; determine a response based at least in part on the determined status of management medication regimen; and transmit the response.

According to another aspect of the present invention, a method of remotely managing a medication regimen in a system including a communication device usable by a target individual, the communication device communicating over a network, at least one peripheral device that obtains biometric information regarding the target individual, the at least one peripheral device communicating over the network to transmit the biometric information, and a medication dispenser usable by the target individual, the medication dispenser communicating over the network to transmit information regarding the medication in the medication dispenser, includes causing the communication device to enable a conversation interface associated with a virtual assistant, the virtual assistant providing at least one of information, queries, and directions to the target individual through the conversation interface; receiving input from the target individual through the conversation interface; receiving the information regarding the medication from the medication dispenser; receiving the biometric information regarding the target individual from the at least one peripheral device; determining a status of management of the medication regimen based on at least one of the input from the target individual, the information regarding the medication from the medication dispenser, and the biometric information regarding the target individual from the at least one peripheral device; determining a response based at least in part on the determined status of management medication regimen; and transmitting the response.

According to yet another aspect of the present invention, a non-transitory, computer-readable medium stores computer-executable instructions that, when executed on one or more processors, cause the one or more processors to perform a method to remotely manage a medication regimen in a system including a communication device usable by a target individual, the communication device communicating over a network, at least one peripheral device that obtains biometric information regarding the target individual, the at least one peripheral device communicating over the network to transmit the biometric information, and a medication dispenser usable by the target individual, the medication dispenser communicating over the network to transmit information regarding the medication in the medication dispenser, the method including causing the communication device to enable a conversation interface associated with a virtual assistant, the virtual assistant providing at least one of information, queries, and directions to the target individual through the conversation interface; receiving input from the target individual through the conversation interface; receiving the information regarding the medication from the medication dispenser; receiving the biometric information regarding the target individual from the at least one peripheral device; determining a status of management of the medication regimen based on at least one of the input from the target individual, the information regarding the medication from the medication dispenser, and the biometric information regarding the target individual from the at least one peripheral device; determining a response based at least in part on the determined status of management medication regimen; and transmitting the response.

These and other aspects, objects, features, and advantages of the invention will become apparent from the following detailed description of illustrative embodiments thereof, which is to be read in connection with the accompanying drawings.

### BRIEF DESCRIPTION OF THE DRAWINGS

FIG. 1 illustrates a system according to an embodiment of the present invention.

FIG. 2 illustrates a flow chart of the process of the present invention including communication between the adherence bot and the target individual.

FIGS. 3A-3D show examples of dialog in the conversation UI between the adherence bot and the target individual.

FIG. 4 shows an example of the structure of an adherence bot according to an embodiment of the present invention.

### DETAILED DESCRIPTION OF THE PREFERRED EMBODIMENTS

This disclosure describes a system, method, and bot for assisting targeted individuals (patients, users) with their healthcare. The techniques described herein provide for a personal virtual healthcare assistant or adherence bot that engages in dialogs with the targeted individual to help with medication adherence and other aspects of healthcare. To facilitate the exchanges between the targeted individual and adherence bot, a conversation user interface (UI) is provided to enable the targeted individual to intuitively understand his or her interactions with the adherence bot.

With the present invention, the adherence bot is enabled to:
interface with various peripheral devices such as a smart medicine dispenser, a blood pressure monitor, a scale, a glucose monitor, a smart cap, a hub, etc.
educate patients on setting up and enrollment of their devices
ensure patients test regularly by sending reminders (i.e., perform outreach to patients on behalf of a practice)
trigger alerts/SMS when target individuals fall out of adherence or when target individuals are outside of a safe measurement range
receive and aggregate data from the peripheral devices and use artificial intelligence to process the data to:
   identify reasons for non-adherence
   assist a physician to adjust the titration of medication, switch medication, or alter therapies
   identify target individuals who may be at risk for low adherence/compliance
   identify side effects
provide feedback from healthcare providers in connection with the target individual's health record.
send SMS alerts regarding device shipment status to ensure target individuals are available to receive their devices.

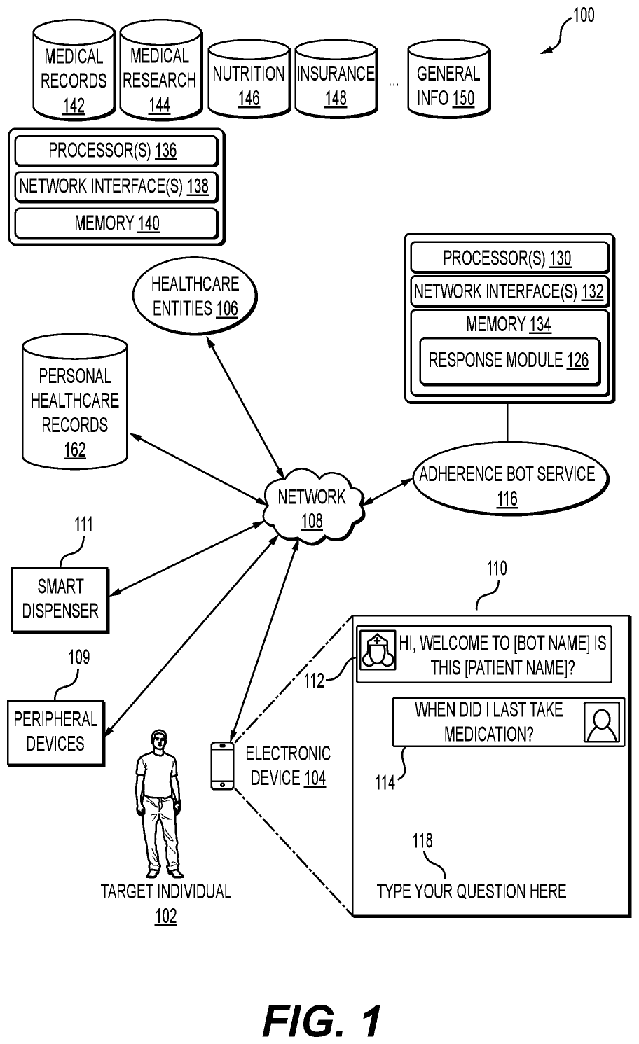

FIG. 1 illustrates a system 100 according to an embodiment of the present invention that includes a target individual (patient, user) 102 operating an electronic device 104, such as a smart phone, to receive content from one or more healthcare entities 106. The content may comprise a website, an intranet site, a downloaded application, or any other platform on which the target individual 102 may access information from the healthcare provider(s) 106. In this example, the target individual 102 accesses the platform over a network 108, which may represent any type of communication network, including a local-area network, a wide-area network, the Internet, a wireless network, a wireless wide-area network (WWAN), a cable television network, a telephone network, a cellular communications network, combinations of the foregoing, and/or the like. The target individual 102 is further affiliated with one or more peripheral devices 109 and a medication dispenser 111, such as a smart cap, that can communicate with the system over network 108. The peripheral devices 109 can obtain biometric information of the target individual 102 and can include at least one of a blood pressure reader, a blood glucose reader, a scale, a pulse oximeter, a sleep monitor, and a central hub, although any number of other devices known to those of skill in the art may be used with the system. The medication dispenser 111 can store medication for use by the target individual 102 and includes sensors to detect when the medication has been dispensed and notification devices to provide notifications to the target individual 102, as is known in the art. While it is preferred that peripheral devices 109 and medication dispenser 111 are “smart” devices that can communicate through the network, such is not required. For example, target individual 102 can manually input information from the peripheral devices 109 and the medication dispenser 111 to the network via the electronic device 104.

While FIG. 1 illustrates the electronic device 104 as a smart phone, the electronic device 104 may comprise any sort of device, such as a desktop computer, a multifunctional device, a laptop computer, a tablet computer, a personal digital assistant (PDA), a dedicated hub, or the like. In each instance, the electronic device 104 may include various additional components, such as one or more output devices (e.g., displays, speakers, etc.), one or more input devices (e.g., a keyboard, a touchscreen, etc.), an operating system, system busses, and the like.

The electronic device 104 renders a conversation user interface (UI) 110 that displays conversation with an adherence bot (virtual-assistant) service 116. The conversation UI 110 may be served from servers of the healthcare provider 106 or servers of the adherence bot service 116.

The conversation UI 110 engages the target individual 102 in a conversation that emulates human conversation. In some cases, the conversation UI 110 may include a virtual assistant that has a human-like personality and persona. The virtual assistant may include an avatar and the conversation UI 110 conveys a visual representation of a conversation between the target individual 102 and the avatar or adherence bot service 116. The conversation UI 110 presents a series of dialog representations 112, 114, such as graphical content bubbles, which are designated as representing dialog from either the target individual 102 or the adherence bot. In this illustration, the target individual-originated dialog representations 114 contain input from the target individual 102 (via text or otherwise) and the device- or avatar-originated dialog representations 112 contain responses from the device or adherence bot. The representations 112, 114 may be visually distinguished in the conversation UI 110 in any known manner to visually convey which entity is associated with the content. The conversation UI 110 may also include an interface area 118 that captures input from the target individual 102, including via typed input, audio, or speech input, as well as touch input and gesture input. Gesture or emotive input may be captured if the electronic device 104 is equipped with a camera or other sensor.

The target individual 102 may enter a query into the interface area 118 of the conversation UI 110. The electronic device 104 transmits this query over the network 108 to the adherence bot service 116. In response, the adherence bot service 116 may identify a response to provide to the target individual 102. The response may be added to a dialog representation of the conversation UI 110.

The adherence bot service 116 may comprise one or more computing devices (e.g., one or more servers) that include or otherwise have access to one or more processors 130, one or more network interfaces 132, and memory 134. The healthcare provider 106 may comprise one or more computing devices (e.g., one or more servers) that include or otherwise have access to one or more processors 136, one or more network interfaces 138, and memory 140, which stores or has access to medical records 142 of the target individual 102, medical research 144, nutrition information 146, insurance information 148, and/or general information 150.

The electronic device 104 of the target individual 102 may include or otherwise have access to one or more processors, one or more network interfaces, and memory, which stores a conversation application for rendering the UI 110 and a healthcare application for providing information from the healthcare provider 106 to the target individual 102. The client application may comprise a browser for rendering a site, a downloaded application provided by the healthcare provider 106, or any other client application configured to output content from the healthcare provider 106. While FIG. 1 illustrates the service provider 106 storing the medical records 142, medical research 144, nutrition information 146, and insurance information 148, in some instances the healthcare application 160 may store some or all of this content locally on the device 104.

The various memories 132, 140, and 156 store modules and data, and may include volatile and/or nonvolatile memory, removable and/or non-removable media, and the like, which may be implemented in any method or technology for storage of information, such as computer-readable instructions, data structures, program modules, or other data. Such memory includes, but is not limited to, RAM, ROM, EEPROM, flash memory or other memory technology, CD-ROM, digital versatile disks (DVD) or other optical storage, magnetic cassettes, magnetic tape, magnetic disk storage, or other magnetic storage devices, RAID storage systems, or any other tangible medium which can be used to store the desired information and which can be accessed by a computing device.

While FIG. 1 illustrates one example arrangement of the system 100, it is to be appreciated that many other arrangements can achieve the desired functions and results. For instance, while FIG. 1 illustrates the healthcare entity 106 as separate from the adherence bot service 116, in some instances some or all of these components may reside in a common location, be spread out amongst multiple additional entities, be located on the electronic device 104, and/or the like.

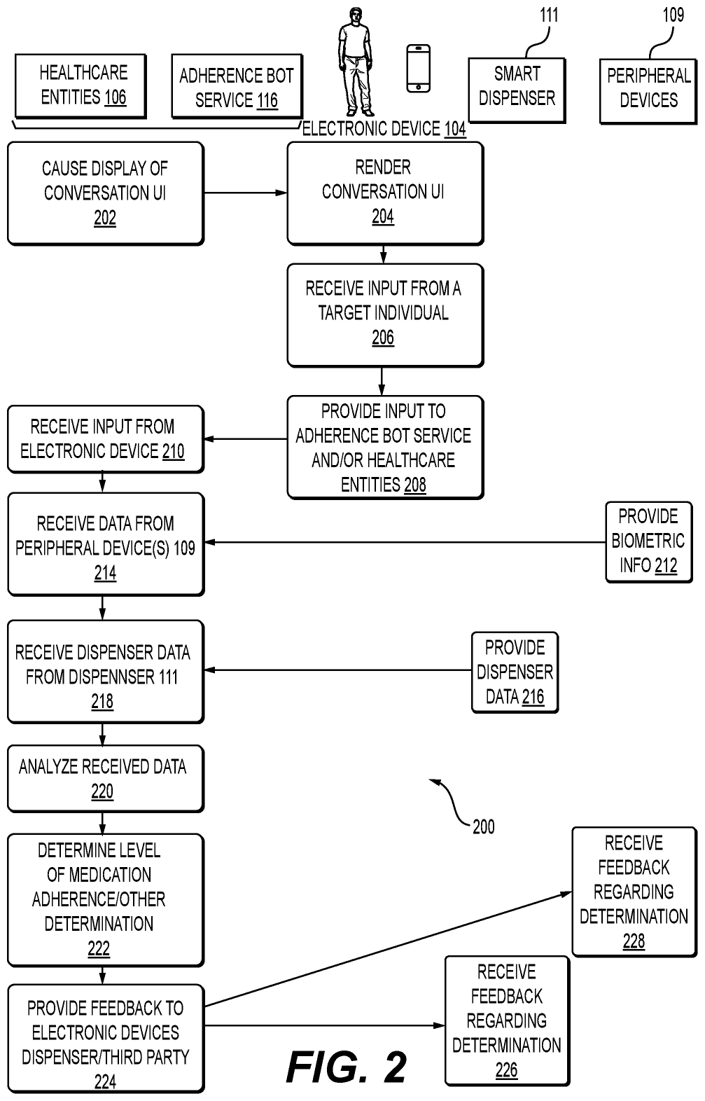

FIG. 2 shows a high-level communication flow 200 between the electronic device 104, peripheral device(s) 109, and medication dispenser 111 associated with the target individual 102 and the healthcare entity 106 and/or the adherence bot service 116. As illustrated, the electronic device 104 renders the conversation UI 110 from the adherence bot service 116. In some instances, the adherence bot service 116 serves the conversation UI 110 to the device 104, while in other instances the healthcare entity 106 serves the conversation UI 110.

The conversation UI 110 emulates human-to-human interaction between the target individual 102 and the healthcare entity 106. The conversation UI 110 includes one or more adherence bot-originated dialog representations associated with the healthcare entity. The adherence bot image may be associated with the healthcare entity or as a personal digital assistant personalized for the target individual 102.

Each query sent to the healthcare entity 106 and/or the adherence bot service 116 may comprise the words and phrases within the string of text entered by the target individual 102, from which concepts may be derived. In some implementations, the concepts may be derived at least partly by the electronic device 104 through some natural language pre-preprocessing. In other implementations, the concepts may be derived as part of the adherence bot service 116 or a combination of the device and service.

A query sent to the healthcare entity 106 and/or the adherence bot service 116 may further comprise one or more pieces of context. The context may be based on any additional factors associated with the target individual 102, the electronic device 104, the peripheral device(s) 109, the medication dispenser 111, or the like. The context may include whether or not the target individual 102 is signed in with the healthcare entity 106, a health status of the target individual 102, an age of the target individual 102, a type of the peripheral device(s) 109 or medication dispenser 111 used by the target individual 102, or the like.

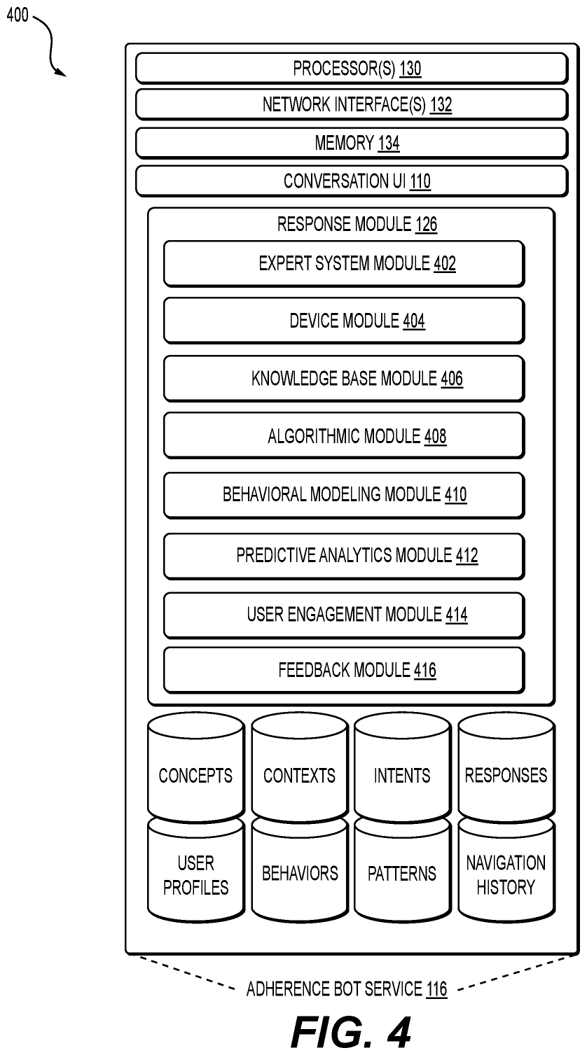

FIG. 4 illustrates example components that the adherence bot service 116 may utilize when determining a response to the target individual's input. As illustrated, the adherence bot service 116 may be hosted on one or more servers that include one or more processors 130, one or more network interfaces 132, and memory 134.

The memory 134 may store or otherwise have access to the conversation UI 110 and a response module 126. The response module 126 may include an expert system module 402, a device module 404, a knowledge base module 406, an algorithmic module 408, a behavior modeling module 410, a predictive analytics module 412, a user engagement module 414, and a feedback module 416. The expert system module 402 employs some combination of machine learning (context aware supervised/semi-supervised or unsupervised) and natural language processing. Suitable applications are available from Rasa and IBM, for example. The device module 404 can interface with devices to gather health/medication related activities. Suitable applications are available from RxCap and Omron. The knowledge base module 406 can interface with medication knowledge databases such as those provided by First DataBank and Epic. The algorithmic module 408 can function as a natural language generation platform, such as Wordsmith from Automated Insights. The behavioral modeling module 410 can tailor messages for specific individuals or groups. This module can combine a machine learning (context aware supervised/semi-supervised or unsupervised) agent with a human.

The predictive analytics module 412 can aid in determining preemptive coaching strategies to prevent reduction of compliance/adherence and may be a combination of any of the above modules. The predictive analytics module 412 can observe target individual activity and attempt to learn characteristics about the target individual that can be used as input to the response module 126. The predictive analytics module 412 may initially access a user profile database to find any preferences that the target individual may have provided. Then, over time, the predictive analytics module 412 may learn any number of characteristics about the target individual, such as health status (generally healthy or very sick), treatment regimens of the target individual (e.g., dialysis, chemotherapy, etc.), a current location of the target individual, insurance eligibility for particular procedures, and the like. In particular, predictive analytics module 412 can learn reasons for the target individual's lack of medication adherence.

The predictive analytics module 412 may also track patterns (e.g., the target individual has been losing weight over a certain time period, the target individual's blood pressure is higher when at work, etc.). Patterns may be stored and each of these observed behaviors, patterns, and navigation history may be useful to the response module 126 by providing additional context to the input of the target individual through the adherence bot 116. Such analysis can be performed through another module.

The user engagement module 414 can utilize other known third-party services to help the user have a more fulfilling experience. The feedback module 416 allows caregivers (and other healthcare entities) to provide feedback to the individual with connection to patient health records to provide a full 360-degree view to the primary care entity. For example, during a dosing protocol, certain side effects may arise they may be harmful to the patient or adversely affect the treatment. Also, the adherence bot may determine that the dosing protocol is not effective and may need to be adjusted or titrated or ceased altogether. This may entail switching medications or other therapy. This can be relayed to the healthcare entity 106, such as a physician, who can then decide whether to provide further instructions to the target individual 102, either in person, over another communication channel (e.g., in a telephone call), or t.

While FIG. 4 illustrates the described modules as residing on the adherence bot service 116, in other instances some or all of these modules may reside in another location. For instance, these modules may reside in whole or part on each of the adherence bot service 116, the healthcare entity 106, the electronic device 104, or at any other location.

FIG. 2 shows an example process 200 that includes the target individual 102 providing a query via the conversation UI 110 and the healthcare entity 106 and/or the adherence bot service 116 determining a response to provide to the target individual 102. This response may take a context of the query into account both when identifying an intent of the query and when identifying an appropriate response. In this example, operations illustrated beneath the electronic device 104 may be performed by this electronic device 104, operations illustrated beneath the peripheral devices 109 may be performed by one or more of these peripheral devices 109, and operations illustrated beneath the medication dispenser 111 may be performed by this medication dispenser 111, while operations illustrated beneath the healthcare entities 106 and the adherence bot service 116 may be performed by the entities and/or the service in some examples. However, it is to be appreciated that in other implementations the operations may be performed at any other location(s).

At 202, the healthcare entities 106 and/or the adherence bot service 116 causes display of the conversation UI on the electronic device 104. The conversation UI may be the sole graphics on a screen, or it may on or adjacent to other content.

At 204, and in response, the electronic device 104 renders the conversation UI 110. At 206, the electronic device 104 receives input from the target individual interacting with the conversation UI. The input may comprise a string of text, verbal input, or some other input (e.g., gesture, video images, etc.). At 208, the electronic device 104 provides the input to the healthcare entities 106 and/or the adherence bot service 116, which receives the input at 210.

At 212, the peripheral device(s) 109 provide acquired biometric information to the healthcare entities 106 and/or the adherence bot service 116, which receives the input at 214. At 216, the medication dispenser 111 provides dispenser data to the healthcare entities 106 and/or the adherence bot service 116, which receives the input at 218. At 220, the healthcare entities 106 and/or the adherence bot service 116 analyze the received information. That is, the healthcare entities 106 and/or the adherence bot service 116 may use language processing and machine learning techniques to identify queries, patterns, behaviors, anomalies, and other information from the received data. In some examples, the concept(s) of a query are determined at least partly with reference to one or more keywords expressed within the input. For instance, the concepts may be determined using relatively basic keyword matching in some instances. This matching can be improved with the adherence bot modules, so that specific words or phrases can be mapped to a given concept based on learned specific user behavior.

At 222, the healthcare entities 106 and/or the adherence bot service 116 may also determine a level of medication adherence, the existence of any side effects, and any adverse drug events, based on the data analyzed at 220. Depending on the adherence level, the existence of any side effects, and any adverse drug events, at 224, the healthcare entities 106 and/or the adherence bot service 116 provides feedback regarding adherence, the existence of any side effects, and any adverse drug events to the electronic device 104 at 226 and/or the medication dispenser 111 at 228. This feedback can also be provided to a third party, such as healthcare entity 106, for example, the target individual's primary care physician. The feedback can include a summary of the analysis, such as the level of medication adherence, the existence of any side effects, and any adverse drug events, as well as recommendations for further treatment including switching, titrating, or ceasing the medication or therapy. This can include recommendations regarding other medications that may be having interactions with the target medication.

Using a combination of the modules, the adherence bot can ensure that specific questions/responses will be used to identify non-adherent users and address the problems. Based on the received and processed data, the adherence bot can:
Identify difficulties and barriers related to adherence (cost, side effects, forgetfulness, etc.)
Address the problems (reminders, medication information, resources)
Inform the target individuals accordingly how the problems have been addressed (provide behavioral support, explain key information, connect them to their doctors/care providers).

The timing of questions is also important. The adherence bot will ask “health related questions” at the same time the target individual is interacting with the medication. Similarly, the peripheral devices 109 can sample the biometric data at this timing to receive relevant data. Because the medication dispenser will have the medication information the user is taking, the system can help monitor for side effects using biometric data (from the hub, other peripheral devices, and/or self-reporting). As medication adherence increases or decreases, the system can update both the doctor and patient on the impact of the adherence levels. For example, a medication might cause a side effect of causing trouble sleeping. The system can monitor the target individual's medication intake as well as the sleep duration automatically and report the findings/correlations back to the user or doctor to help them make a health valuation on the impact. Another example is with regard to mental health surveys, such as PHQ-9, which includes a set of several patient questions. These questions can be sent through the adherence bot at the right time (such as when a smart cap having antidepressant medication is opened) and biometric data (lack of sleep, etc.) can be analyzed. If the analysis results in a high alert, for example, the adherence bot can escalate to alert a provider/family member/suicide hotline, etc.

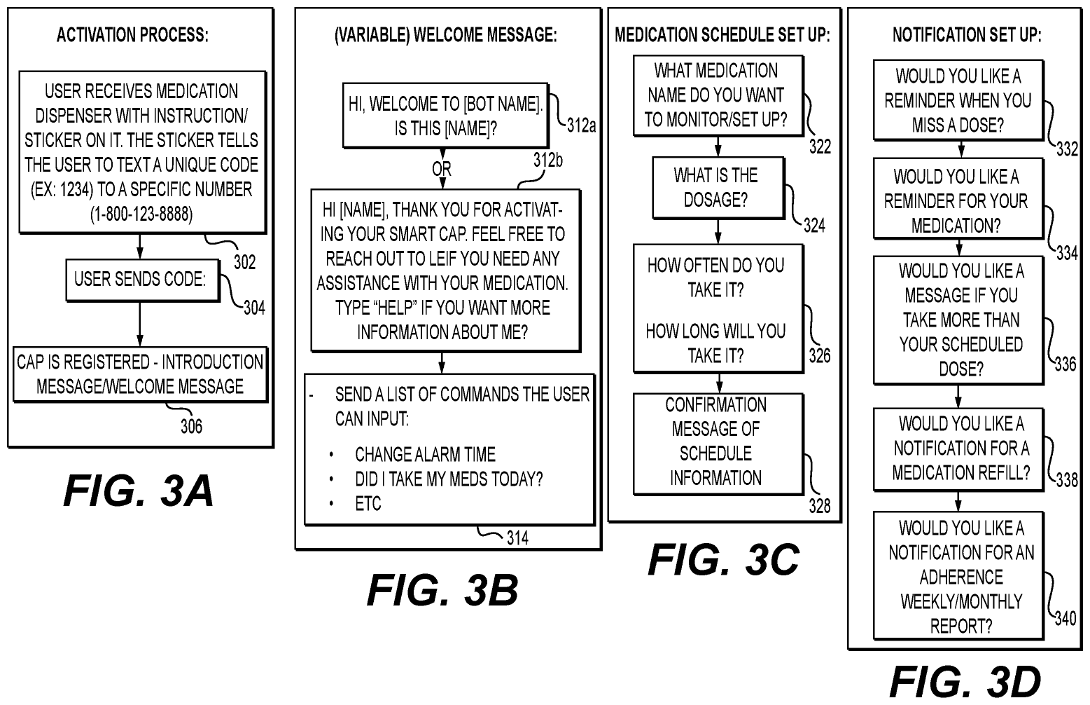

The adherence bot can initiate several processes in its dialog with the target individual 102. Some of these processes are shown in FIGS. 3A-3D. 3A shows a sample activation process. For example, when receiving a new medication dispenser 111, the target individual 102 can read a sticker provided with the unit in 302 and text a unique code to a particular text number in 304. After the dispenser is registered, the adherence bot can issue an introduction message in the dialogue in 306. Examples of the welcome message are shown in 312a and 312b in FIG. 3B. The adherence bot can also send a list of commands or features that the target individual 102 can input in 314. Examples of such commands and features are as follows:

Glow: A user can text “Glow” or similar command into the electronic device to prompt the medication dispensers, such as a smart caps, to light up the caps corresponding to the medications that are due to be taken. The smart caps can have memory that stores the scheduling information.

Location: Shows the user via the screen of the electronic device Where the medication dispenser is located.

Enhanced Security: Allows a user to receive a text message each time the cap is opened.

Double Dose Alert: Alerts the user if the user opens the cap after the initial dose was due, i.e., alerts the user if the medication dispenser was accessed already.

Pharmacist—Links a user with a pharmacist or live nurse/caregiver.

Caregiver/Group Monitoring—Allows a user to add an individual to the SMS chat to provide access to see when the user has taken his or her medication (e.g., friends/family).

Change Schedule: Allows a user to adjust the reminders and medication schedule.

Change Medication: Allows a user to change the medication name.

Missed: Allows a user to see what medications were missed that day.

Help—Sends a list of commands and how to use the adherence bot.

Battery level—Sends battery reading of the medical dispenser.

Did I take my meds/Last take—Sends information regarding when the user last took the medication.

Medication info—Sends medication information from third parties.

Side effects—Sends side effect information regarding the medication the user is taking.

Drug to drug interactions—User can ask if a medication interacts with the current medication being taken.

Education—Sends relevant drug information to the user.

Chime—Causes the medication dispenser to make an audible sound

The adherence bot can also set up the medication schedule with the target individual 102 as shown in FIG. 3C. In 322-326, the adherence bot asks a series of questions regarding the medication. After receiving the requested data, the adherence bot issues a confirmation message regarding the schedule information in 328. The adherence bot is also programmed to issue notifications to the target individual 102. These notifications can either be issued through electronic device 104 or the medication dispenser 111. In 332-340, the adherence bot asks a series of questions regarding reminders for taking the medication, refilling the medication, and receiving monthly reports.

Although this invention has been described in certain specific exemplary embodiments, many additional modifications and variations will be apparent to those skilled in the art in light of this disclosure. It is, therefore, to be understood that this invention may be practiced otherwise than as specifically described. Thus, the exemplary embodiments of the invention should be considered in all respects to be illustrative and not restrictive, and the scope of the invention to be determined by any claims supportable by this application and the equivalents thereof, rather than by the foregoing description.

What is claimed:

1. A system for remotely managing a medication regimen, comprising:
a communication device usable by a target individual, the communication device communicating over a network;
at least one peripheral device that obtains biometric information regarding the target individual, the at least one peripheral device communicating over the network to transmit the biometric information;
a medication dispenser usable by the target individual, the medication dispenser communicating over the network to transmit information regarding the medication in the medication dispenser; and
at least one processor communicating with the communication device, the at least one peripheral device, and the medication dispenser over the network to execute computer-executable instructions to:
cause the communication device to enable a conversation interface associated with a virtual assistant, the virtual assistant providing at least one of information, queries, and directions to the target individual through the conversation interface;
receive input from the target individual through the conversation interface;
receive the information regarding the medication from the medication dispenser;
receive the biometric information regarding the target individual from the at least one peripheral device;
determine a status of management of the medication regimen based on at least one of the input from the target individual, the information regarding the medication from the medication dispenser, and the biometric information regarding the target individual from the at least one peripheral device;
determine a response based at least in part on the determined status of management medication regimen; and
transmit the response.

2. The system according to claim 1, wherein the conversation interface allows communication between the target individual and the virtual assistant using at least one of text format including Short Message Service (SMS), audible voice, and image graphics.

3. The system according to claim 1, wherein the response comprises a message to the target individual from the virtual assistant through the conversation interface.

4. The system according to claim 1, wherein the response comprises a signal sent to the medication dispenser.

5. The system according to claim 1, wherein a third party is in communication with the at least one processor over the network and the response comprises a message to the third party.

6. The system according to claim 5, wherein the message to the third party includes the status of management medication regimen for the target individual and the at least one processor allows the third party to instruct the virtual assistant to contact the target individual regarding adjusting the medication regimen.

7. The system according to claim 1, wherein the status of management of the medication regimen includes information regarding adherence to the medication regimen by the target individual.

8. The system according to claim 7, wherein the at least one processor determines adherence to the medication regimen by the target individual using analysis of aggregated data.

9. The system according to claim 8, wherein the aggregated data includes inputs from the target individual through the conversation interface, the information regarding the medication from the medication dispenser, the biometric information regarding the target individual from the at least one peripheral device, and general population data including medical research results, nutrition information, and insurance information.

10. The system according to claim 7, wherein the information regarding adherence to the medication regimen by the target individual includes at least one of identification of reasons for non-adherence, information to assist a third party in adjusting titration of the medication, identification of target individuals who may be at risk for low adherence, and identification of side effects to the medication.

11. The system according to claim 1, wherein the at least one peripheral device that obtains biometric information includes at least one of a blood pressure reader, a blood glucose reader, a scale, a sleep monitor and a pulse oximeter.

12. A method of remotely managing a medication regimen in a system including a communication device usable by a target individual, the communication device communicating over a network, at least one peripheral device that obtains biometric information regarding the target individual, the at least one peripheral device communicating over the network to transmit the biometric information, and a medication dispenser usable by the target individual, the medication dispenser communicating over the network to transmit information regarding the medication in the medication dispenser, the method comprising:
causing the communication device to enable a conversation interface associated with a virtual assistant, the virtual assistant providing at least one of information, queries, and directions to the target individual through the conversation interface;
receiving input from the target individual through the conversation interface;
receiving the information regarding the medication from the medication dispenser;
receiving the biometric information regarding the target individual from the at least one peripheral device;
determining a status of management of the medication regimen based on at least one of the input from the target individual, the information regarding the medication from the medication dispenser, and the biometric information regarding the target individual from the at least one peripheral device;
determining a response based at least in part on the determined status of management medication regimen; and
transmitting the response.

13. The method according to claim 12, wherein the conversation interface allows communication between the target individual and the virtual assistant using at least one of text format including Short Message Service (SMS), audible voice, and image graphics.

14. The method according to claim 12, wherein the response comprises a message to the target individual from the virtual assistant through the conversation interface.

15. The method according to claim 12, wherein the response comprises a signal sent to the medication dispenser.

16. The method according to claim 12, wherein the response comprises a message to a third party.

17. The method according to claim 16, wherein the message to the third party includes the status of management of the medication regimen for the target individual and the third party is permitted to instruct the virtual assistant to contact the target individual regarding adjusting the medication regimen.

18. The method according to claim 12, wherein the status of management of the medication regimen includes information regarding adherence to the medication regimen by the target individual.

19. The method according to claim 18, wherein adherence to the medication regimen by the target individual is determined using analysis of aggregated data.

20. The method according to claim 19, wherein the aggregated data includes inputs from the target individual through the conversation interface, the information regarding the medication from the medication dispenser, the biometric information regarding the target individual from the at least one peripheral device, and general population data including medical research results, nutrition information, and insurance information.

21. The method according to claim 18, wherein the information regarding adherence to the medication regimen by the target individual includes at least one of identification of reasons for non-adherence, information to assist a third party in adjusting titration of the medication, identification of target individuals who may be at risk for low adherence, and identification of side effects to the medication.

22. The method according to claim 12, wherein the at least one peripheral device that obtains biometric information includes at least one of a blood pressure reader, a blood glucose reader, a scale, a sleep monitor, and a pulse oximeter.

23. A non-transitory, computer-readable medium storing computer-executable instructions that, when executed on one or more processors, cause the one or more processors to perform a method to remotely manage a medication regimen in a system including a communication device usable by a target individual, the communication device communicating over a network, at least one peripheral device that obtains biometric information regarding the target individual, the at least one peripheral device communicating over the network to transmit the biometric information, and a medication dispenser usable by the target individual, the medication dispenser communicating over the network to transmit information regarding the medication in the medication dispenser, the method comprising:
causing the communication device to enable a conversation interface associated with a virtual assistant, the virtual assistant providing at least one of information, queries, and directions to the target individual through the conversation interface;
receiving input from the target individual through the conversation interface;
receiving the information regarding the medication from the medication dispenser;
receiving the biometric information regarding the target individual from the at least one peripheral device;
determining a status of management of the medication regimen based on at least one of the input from the target individual, the information regarding the medication from the medication dispenser, and the biometric information regarding the target individual from the at least one peripheral device;
determining a response based at least in part on the determined status of management medication regimen; and
transmitting the response.
